(19) United States
(12) Patent Application Publication
(10) Pub. No.: US 2023/0220370 A1
(43) Pub. Date: Jul. 13, 2023

(54) Programming Living Glue Systems to Perform Autonomous Mechanical Repairs

(71) Applicant: ShanghaiTech University, Shanghai (CN)

(72) Inventors: Chao Zhong, Shanghai (CN); Bolin An, Shanghai (CN); Yanyi Wang, Shanghai (CN); Xiaoyu Jiang, Shanghai (CN)

(73) Assignee: ShanghaiTech University, Shanghai (CN)

(21) Appl. No.: 18/178,554

(22) Filed: Mar. 6, 2023

Related U.S. Application Data
(63) Continuation of application No. PCT/CN2021/112745, filed on Aug. 16, 2021.

(30) Foreign Application Priority Data
Sep. 9, 2020 (WO) PCT/CN2020/114171
Aug. 16, 2021 (WO) PCT/CN2021/112745

(51) Int. Cl. C12N 11/04; C12N 15/70; C12N 15/63; C12N 9/02; C07K 14/435; C09J 189/00; C07K 14/245

(52) U.S. Cl. CPC C12N 11/04; C12N 15/70; C12N 15/635; C12Y 114/18001; C12N 9/0071; C07K 14/43504; C09J 189/00; C07K 14/245; C12N 2800/101

(57) ABSTRACT

A living engineered glue system for performing autonomous mechanical repairs comprises a biofilm of microbial cells embedded in an extracellular matrix and operably linked in an environmentally-inducible, cell-cell communication genetic circuit to control gene expression.

### INTRODUCTION

Recent research efforts at the interface between biomaterials and bioengineering have resulted in the emergence of a research field exploring engineered living materials (ELMs).¹⁻³ ELMs can harness the power of cellular machinery to synthesize useful materials with different functional properties and can, in theory, recapitulate or reconstitute—in artificial materials—the distinct living dynamic and autonomous features of biological systems, including environmental responsiveness, self-healing, self-replication, remodeling and the capacity for adaptive evolution.^{1,4} The development of ELMs, therefore, represents a new paradigm of materials performance and synthesis that has significant technological implications for the design of future smart and/or autonomous materials. ELMs demonstrated to date include the deployment of microbes to produce functional biofilms for various usages,⁵⁻⁸ to fabricate self-reproducing building bricks,⁹ to harvest bacterial cellulose production, ^{10,11} to act as environmental biosensors,^{12,13} to degrade pollutants,¹⁴ and to act as acoustic reporters for tumor localization,¹⁵ among several other innovative applications.¹⁶⁻¹⁹ Despite these important advances, it remains elusive to adapt current ELMs for performing on-demand mechanical operations, and for accomplishing autonomous repair tasks as natural living systems do. Tackling these technical challenges requires a strategy that effectively and efficiently exploits the living dynamic and autonomous features of state-of-the-art ELMs.

Myriad tools developed by synthetic biologists now enable robust engineering of cells with increasingly complex genetic circuits to sense biological inputs and control gene expression.^{20,21} The integration of engineered tools from synthetic biology and materials science will thus likely lead to the development of living materials with ever-more-sophisticated dynamic functionalities and higher levels of autonomy. We previously demonstrated living cellular glue: these ELMs comprise Bacillus subtilis biofilms engineered with adhesive mussel foot proteins, and they exhibited underwater adhesion and strong environmental tolerance.²² However, beyond consideration of living glue materials performance per se, we have been further motivated by the way that marine organisms use their proteinaceous adhesive materials to implement mechanical work,^{23,24} for instance with the striking process through which sandcastle worms build their sophisticated tubular dwellings by gluing bits of sand and seashells together with proteinaceous adhesives (FIG. 1A).²⁵

### SUMMARY OF THE INVENTION

The invention provides materials and methods relating to engineered smart living biofilm glues to perform on-demand mechanical operations. By rationally designing and combining diverse genetic circuits, we develop chemical- and light-regulated living glue systems capable of accomplishing tasks, including the capture of microspheres from solution to form living composite coatings and light-regulated spatially targeted damage repair. Moreover, we demonstrate a living glue system for autonomous repairing: upon sensing blood leaking from a purpose defect, the two bacterial strains comprising this living glue system localize to the damaged site, communicate via a cell-cell communication network, and plug the leak with their amyloid glue components. The developed methods enable engineered living materials (ELMs), including smart glues for autonomous repairs in both industrial and medical settings, with dynamic, self-healing, and other previously unattainable material properties, with applications such as river pathogens-killing, gastrointestinal diseases-treating, and corrosion's prevention.

In an aspect the invention provides a living engineered glue system for performing autonomous mechanical repairs, the system comprising: a biofilm of microbial cells embedded in an extracellular matrix and operably linked in an environmentally-inducible, cell-cell communication genetic circuit to control gene expression, the cells comprising (a) a glue-producing strain secreting a signal molecule and expressing a fusion protein comprising an adhesive domain and a biofilm protein domain, wherein expression of the fusion protein is induced by an environmental inducer; and (b) an adhesion enhancing strain expressing a tyrosinase, wherein expression of the tyrosinase is induced by the signal molecule secreted by the glue-producing strain.

In embodiments:

the adhesive domain is selected from a marine organism protein adhesive domain (such as a mussel foot protein domain or a barnacle amyloid adhesive domain), a metal-binding peptides/protein domain, minerals-binding peptide/protein domain, and a trefoil factor family (TFF) protein domain;

the adhesive domain comprises a mussel foot protein domain selected from a Mfp3, Mfp3s, Mfp5, Mfp8, and Mfp3s-derived peptide;

the biofilm is selected from an E. coli biofilm (CsgA-based), a B. subtilis biofilm (TasA-based), a kombucha biofilm (acetic acid bacteria (Acetobacteraceae) and osmophilic yeast), and a yeast biofilm (Sup35 amyloid protein-based);

the biofilm protein domain is selected from: TasA (B. subtilis), CsgA (E. coli), PSMs (S. aureus), RmbC (V. cholera), CsgA (Enterobacter cloacae), FapC (Pseudomonas spp.), CsgA (Salmonella spp.) or PAc (Streptococcus mutans);

the biofilm protein domain comprises a CsgA monomer;

the environmental inducer is selected from a blood component (e.g., heme), light (e.g., blue/red/green light), heat/thermal, salt/electrolyte concentration, pH, electrons, and small signal molecules such as isopropyl-beta-D-thiogalactoside (IPTG), anhydrotetracycline (aTC), bile acid or thiosulfate;

the genetic circuit provides a sensor for, and is environmentally-responsive to a signal selected from: aTc/blue light, green/red light, blood/heme, thermal/heat, pH, salt concentration, IPTG, bile acid, thiosulfate and electrons; and/or

the microbial cells are selected from: Bacillus spp. (e.g. B. subtilis), Pseudomonas spp. (e.g. P. aeruginosa), Staphylococcus spp. (e.g. S. aureus), Salmonella ssp. (e.g. S. enterica), Vibrio spp. (e.g. V. cholera), Streptococcus spp. (e.g. Streptococcus mutans), Enterobacter spp. (e.g. Enterobacter cloacae), Lactobacillus spp. (e.g. L. plantarum) or Escherichia spp. (e.g. E. coli).

In an aspect the invention provides a method of using a subject living glue system for performing autonomous mechanical repairs to a surface of a mechanical device or component thereof, such as sealing a defect, comprising the step of: providing the surface coated with the system or applying the system to the surface, under conditions wherein the system autonomously senses and repairs the defect.

In an aspect the invention provides a method of using a subject living glue for performing autonomous mechanical repairs to a surface of a mechanical device or component thereof, such as sealing a defect, comprising the step of: under conditions wherein the system autonomously senses and repairs the defect.

In an aspect the invention provides a method of making a subject living glue comprising engineering and/or combining the glue-producing strain and the adhesion enhancing strain to form the system.

The invention includes all combinations of recited particular embodiments as if each combination had been laboriously recited.

### BRIEF DESCRIPTION OF THE FIGURES

FIG. 1. Biologically inspired design of environmentally responsive living glue systems that enable spatially targeted or autonomous repairs. Schematic for spatially targeted or autonomous mechanical repairs using environmentally responsive cellular glue composed of living biofilm system with engineered and post-translationally modified fusion proteins combining amyloid nanofibers and adhesive peptides from marine mussels.

FIGS. 2A-2I. Design and characterization of an a chemically induced living glue system and its enabled composite coating. (A) Schematic showing the design of aTc-induced living glue system and its integrated gene circuits. The dual-strain living glue system included a glue-producing strain that inducibly produced CsgA-Mfp3s and constitutively produced AHL (aTc_{Receiver}/CsgA-Mfp3s+AHL_{Sender}) and an adhesion-enhancing strain under regulation by an AHL-inducible riboregulator (AHL_{Receiver}/Tyrosinase). The two strains communicate via the diffusible cellular communication signal, AHL. (B) TEM images, Crystal violet (CV) staining, and Congo red (CR) assay showing the massive amounts of protein adhesion components produced by the engineered bacteria as triggered with the aTc inducer. (C) Melanin detection and Nitro Blue Tetrazolium (NBT) assays revealed successful secretion of active tyrosinase from the receiver strain. (D) Schematic illustration of a colloidal AFM probe (R=10 μm for gold tips) used to measure asymmetric adhesion of nanofibers. The graph on the right is a representative AFM image showing marked bacteria and adhesive amyloid nanofibrils. (E) Representative force curves measured by gold-coated (d=20 μm) probes. A single force model was performed for all the experiments. The X-axis (ZSnsr) represents the displacement between the sample surface and the resting position of the cantilever. (F) Comparison of adhesive performance for the different living glue systems. (G) Schematic illustrating the use of the living glue system to capture non-sticky microsphere filler materials to form composite coatings. (H) Representative fluorescent images and scanning electron microscopy (SEM) images for adhered microspheres. (I) The amount of retained microspheres after washing counted for the aTc-induced dual-strain system and a non-induced control.

FIGS. 3A-3G. Design and characterization of a blue light-sensing living glue system for spatially targeted damage repair. (A) Schematic illustrating the blue light-regulated living glue system and its integrated gene circuits. The dual-strain living glue system includes a glue-producing strain that inducibly produces CsgA-Mfp3s in the presence of blue light and constitutively produces AHL (Light_{Receiver}/CsgA-Mfp3s+AHL_{Sender}) and an adhesion-enhancing strain under regulation by an AHL-inducible riboregulator (AHL_{Receiver}/Tyrosinase). The two strains communicate via the diffusible cellular communication signal, AHL. (B) Patterned production of living glue in liquid mediums regulated by programmed blue light (470 nm), which emitted from a commercial digital projector (Model: Aodian-M19, China). (C) Crystal Violet staining was used to visualize the cellular glue pattern. (D) AFM morphology of this living cellular glue with or without light exposure. (E) Patterned assembly of microspheres using the light-inducible dual-strain glue system to capture microspheres from solution in a precise spatially regulated way. (F) Schematic illustrating a living-glue-enabled targeted repair system upon light exposure to specific damage sites, the light-sensing glue system responded by expressing adhesive protein components that eventually glued microspheres together and filled the cracks in the crevice. (G) Practical demonstration of successful repairs characterized using fluorescent microscopy and scanning electron microscopy.

FIGS. 4A-4H. Design and characterization of a blood-sensing living glue system for autonomous damage repair. (A) Schematic illustrating the blood-induced living glue system and its integrated gene circuits. The living glue system includes two strains: a fast glue-producing strain harboring a heme-sensitive gene circuit that inducibly produces CsgA-Mfp3s in the presence of blood and constitutively produces AHL (Heme_{Receiver}/CsgA-Mfp3s+AHL_{Sender}+CsgBCEFG), and an adhesion-enhancing strain under regulation by an AHL-inducible riboregulator (AHL_{Receiver}/Tyrosinase). The two strains communicate via the diffusible cellular communication signal, AHL. (B) Blood dose-response of this living glue system. Each column represents relative glue biomass induced at different blood concentrations based on three independent biological replicates measured by Crystal violet staining (corresponding digital images of Crystal violet-stained samples were presented above (C) TEM images showing the massive amounts of protein adhesion components produced from the engineered bacteria triggered by the blood. (D) Schematic for the autonomous repair of targeted damage (a porous membrane embedded inside a damaged microfluidic device). A small hole (d=1.6 mm) was purposely chiseled on the microfluidic pipe and was then covered with a porous aluminum membrane filter. The pores (d=400 nm) on the membrane would prevent bacteria from entering the microfluidic channel but allow heme molecules to diffuse through, thus site-specifically triggering surrounding bacteria to produce protein adhesion components. (E) SEM images of the repair achieved autonomously by the blood-sensing living glue system operating at different blood flow rate (Q). White circles represent defect areas/sites on the formed glue coatings. (F) Fluorescence leakage experiments for assessing the repair effects. Q1(2, 3, 4, 5) refers to repairing under different blood flow ranging from 10 to 120 μL/h as described in E. (G) Fluorescence leakage experiments (operating at a blood flow of 10 μL/h) and corresponding SEM images (inserts) showing the damage repairs achieved by the two blood-sensing living glue systems. (H) Comparison of the burst pressure resistance for the two different types of living glues.

FIGS. 5A-5D. Plasmid maps of CsgA-Mfps constructed for the living cellular glue systems. (A), Schematic showing the design of aTc-regulated living glue composed of CsgA-Mfps proteins. (B) aTc-inducible CsgA-Mfp3s. (C) aTc-inducible CsgA-Mfp3. (D) aTc-inducible CsgA-Mfp5.

FIGS. 6A-6C. Plasmid maps of a dual-strain living cellular glue system enabled by a cell-cell communication genetic circuit. (A) Schematic showing the design of the aTc-regulated dual-strain living glue system. (B) The plasmid map of the glue-producing strain encoding aTc-inducible expression of CsgA-Mfp3s protein and AHL molecules. (C) The plasmid map of the adhesion-enhancing strain encoding the AHL-inducible expression of OsmY-tyrosinase.

FIGS. 7A-7C. Plasmid maps of a light-regulated dual-strain living cellular glue system enabled by a cell-cell communication genetic circuit. (A), Schematic showing the design of a light-regulated dual-strain living glue system. (B) The plasmid map of the glue-producing strain encoding the blue light-regulated expression of CsgA-Mfp3s protein and AHL molecules. (C) The plasmid map of the adhesion-enhancing strain encoding AHL-inducible expression of OsmY-tyrosinase was used in this system.

FIGS. 8A-8E. Plasmid maps of a blood-sensing dual-strain living cellular glue system enabled by a cell-cell communication genetic circuit. (A) Schematic showing the design of the blood/heme-sensing dual-strain living glue system. (B) The plasmid map of pJFR-ChuA, which encodes a constitutively expressed ChuA protein and a core fragment of T7 RNA polymerase (T7 core). (C) The plasmid map of pJFR-HtrR, which constitutively expresses a heme-sensitive transcriptional repressor HtrR and a heme-induced σ fragment. (D) The plasmid map of the glue-producing strain encoding recombinant CsgA-Mfp3s and AHL molecules expressed under the tight control of intact T7 RNA polymerase. (E) The plasmid map of the adhesion-enhancing strain encodes the expression of AHL-inducible OsmY-Tyrosinase used in this system. Note that the two plasmids shown in (B) and (C) were all transformed in the blood-sensing dual-strain system.

FIG. 9. The design of a burst pressure setup for assessing the adhesion robustness of the living glue systems. A pressure/flow controller (Model No: PG-MFC-8CH, Precigenome, USA) was used to apply pressure on the biofilm glues, and the burst pressure values are correspondingly recorded once the enclosed fluorescent solution released out from the microfluidic channel.

### DESCRIPTION OF PARTICULAR EMBODIMENTS OF THE INVENTION

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

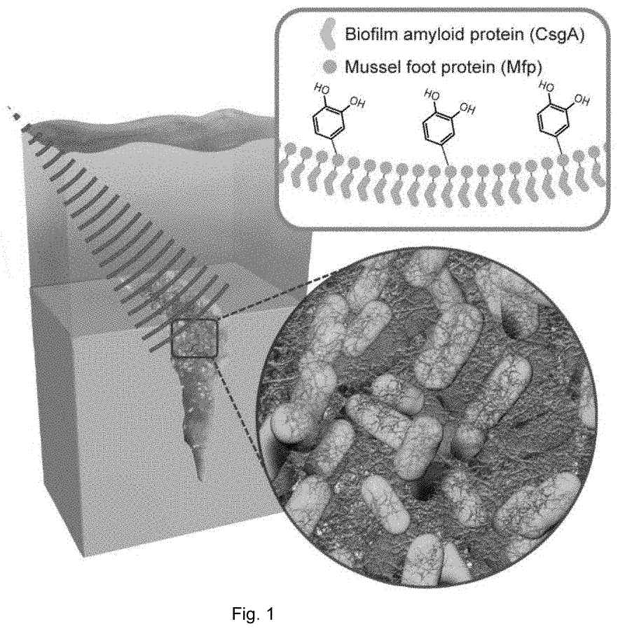

Starting with the successful development of Escherichia coli (E. coli) glue, we rationally designed and combined diverse genetic circuits to create environmentally responsive living glue systems (FIG. 1B). We show that our living glue systems are capable of predictably implementing dynamic on-demand underwater adhesion and even accomplishing autonomous repair tasks.

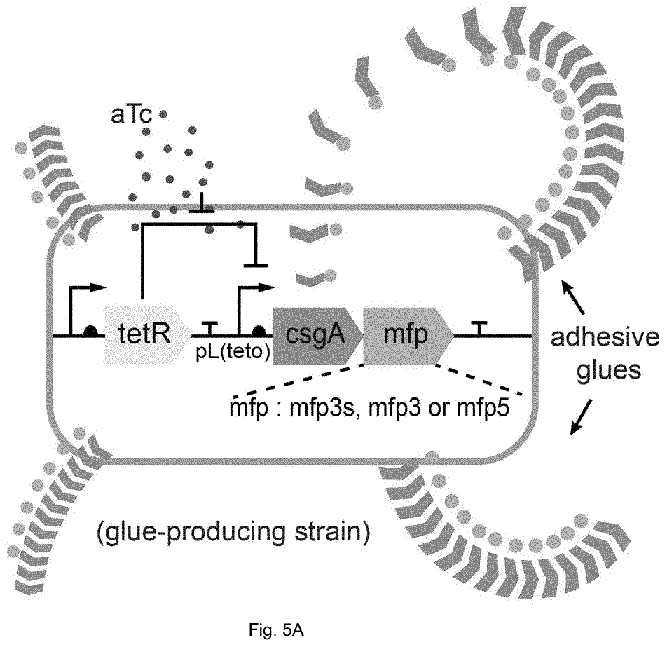

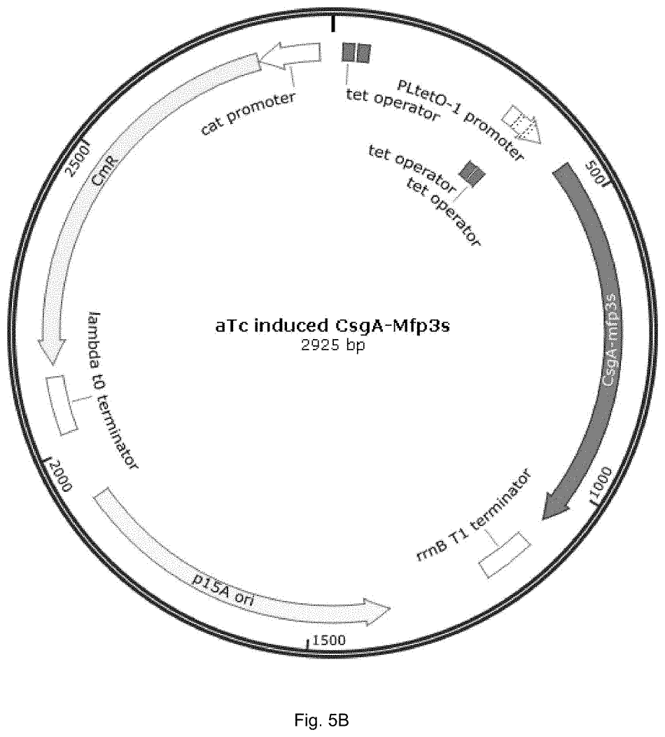

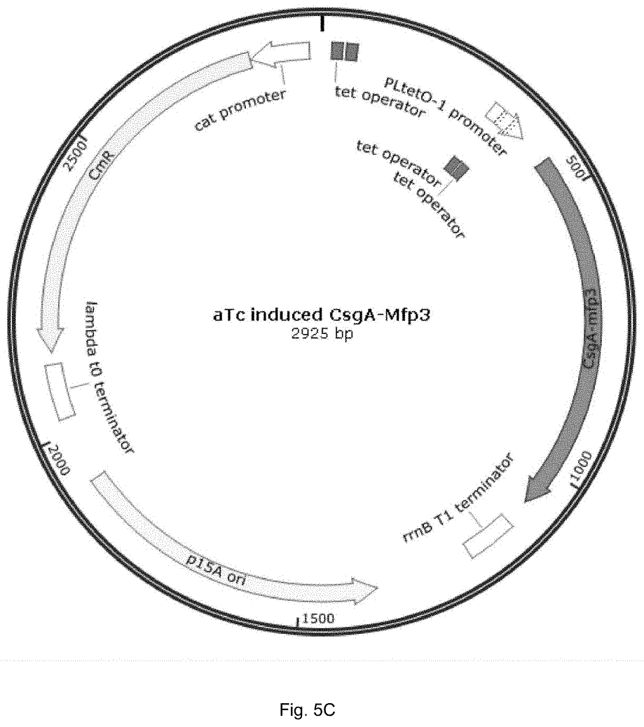

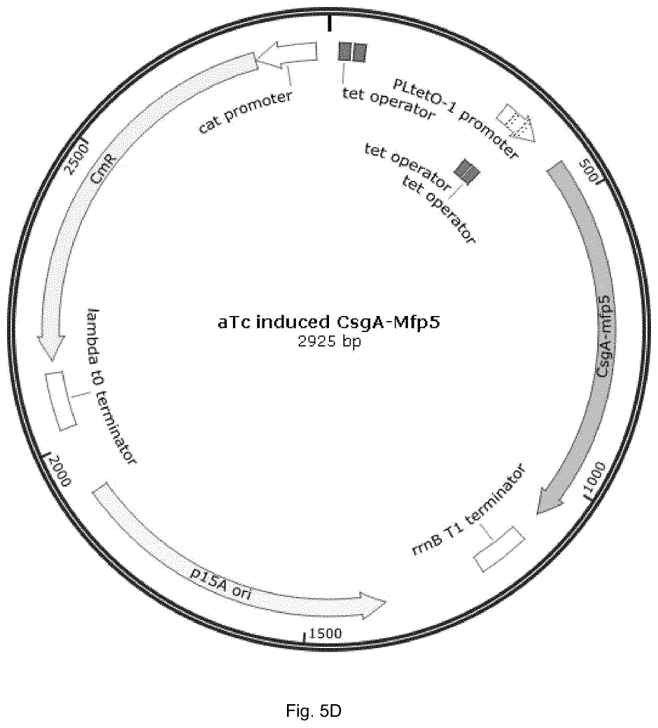

As there are abundant stimuli-responsive genetic circuits available for the engineering of E. coli,²¹ we initially focused on generating suitable E. coli-based living glue to use in ELM systems for autonomous mechanical work (FIG. 1B). Previous in vitro work showed that the curli amyloid fibrils composed of CsgA monomer fused to mussel foot proteins (Mfp) could function as strong underwater adhesives.²⁶ We therefore firstly tested whether the fusion of CsgA with Mfp3 (5.1 kDa), Mfp5 (8.1 kDa), or Mfp3s (5.0 kDa)²⁴ (Table 51) could be functionally generated and secreted by E. coli. The three fusion constructs were introduced into appropriate E. coli host strains separately, and their corresponding protein expressions were tightly regulated by an anhydrotetracycline (aTc)-responsive riboregulatory (FIGS. 5A-5D).⁵ Congo red (CR) staining, Crystal violet (CV) assay, and transmission electron microscopy (TEM) imaging all showed that, among the three designs, the CsgA-Mfp3s fusion protein could massively be secreted and assembled into amyloid adhesive nanofibrils.

Furthermore, the atomic force microscopy (AFM) colloidal probe technique²⁷ showed that the CsgA-Mfp3s biofilm exhibited substantially enhanced adhesion compared with CsgA control. Owing to the presence of the mussel derived fusion domain, the recombinant glue containing CsgA-Mfp3s also showed much-improved adhesion after tyrosinase post-modification (with normalized adhesion force increased from 37.8 mN/m to 126.9 mN/m). This observation was consistent with previous findings that tyrosinase-catalyzed oxidation of tyrosine into 3,4-dihydroxyphenylalanine (Dopa) remarkably improved underwater adhesion of amyloid nanofibrils.²⁸ As Dopa could significantly improve adhesion performances, we, therefore, engineered an AHL-inducible gene construct for tyrosinase (MelC 2) expression²⁹ (fused with osmotically inducible protein Y (Osm Y) for facile secretion³⁰ and co-expressed with its chaperone MelC1 for full enzymatic functions) into the same strain that simultaneously produced the CsgA-Mfp3s fusion components. However, the engineered strains exhibited poor adhesion and failed to attach to substrates under living glue culture conditions. CV and CR staining further revealed that the microbes could not produce a sufficient amount of CsgA-Mfp3s glue components while performing simultaneous expression of tyrosinase, perhaps due to excessive metabolic burden or undesirable intracellular crosslinking.

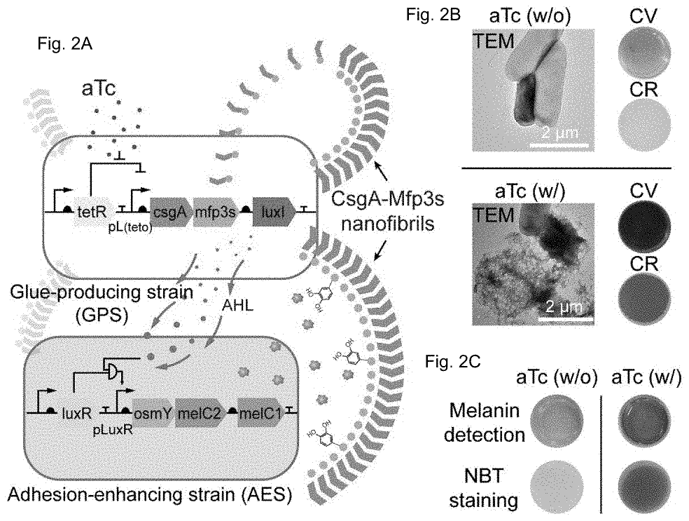

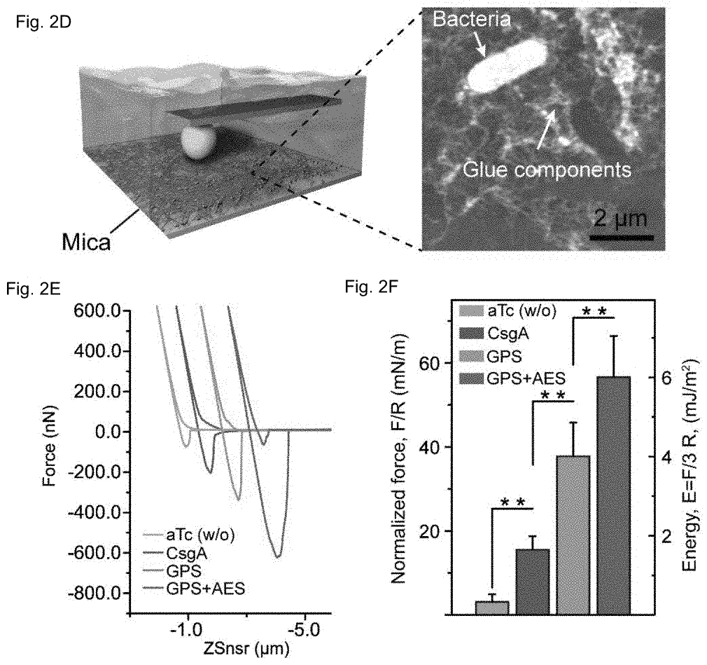

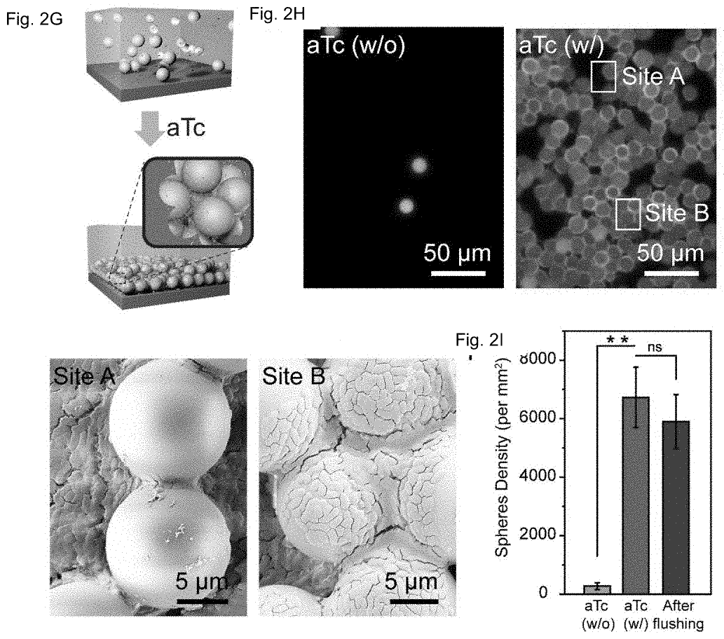

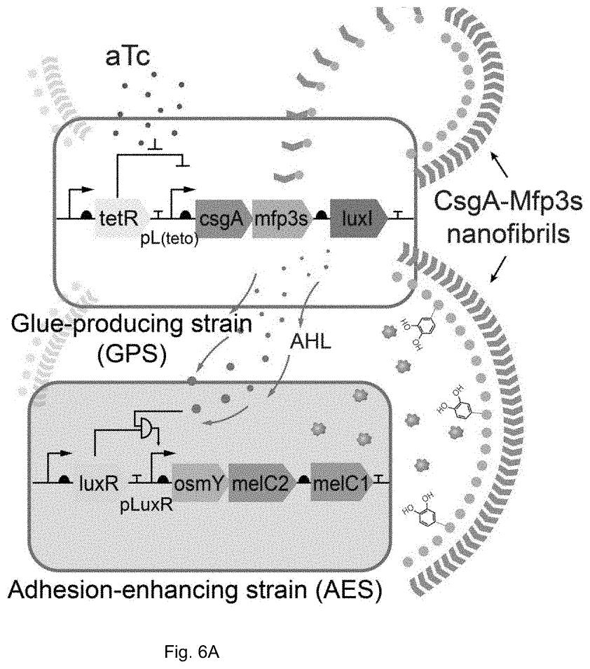

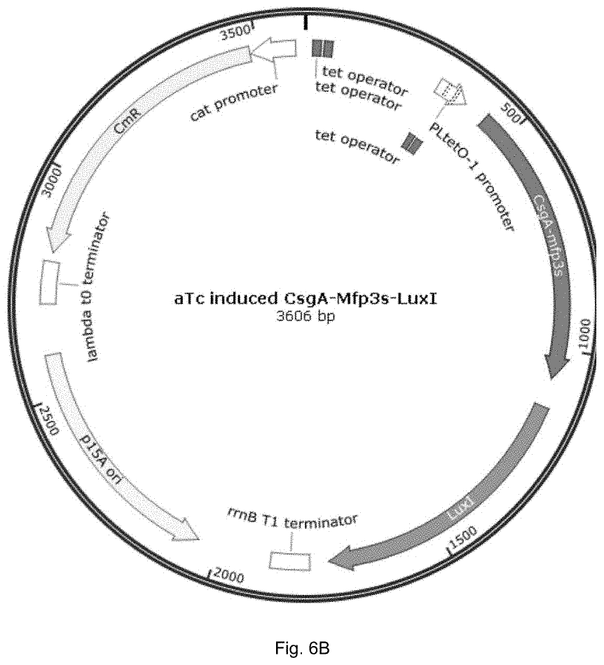

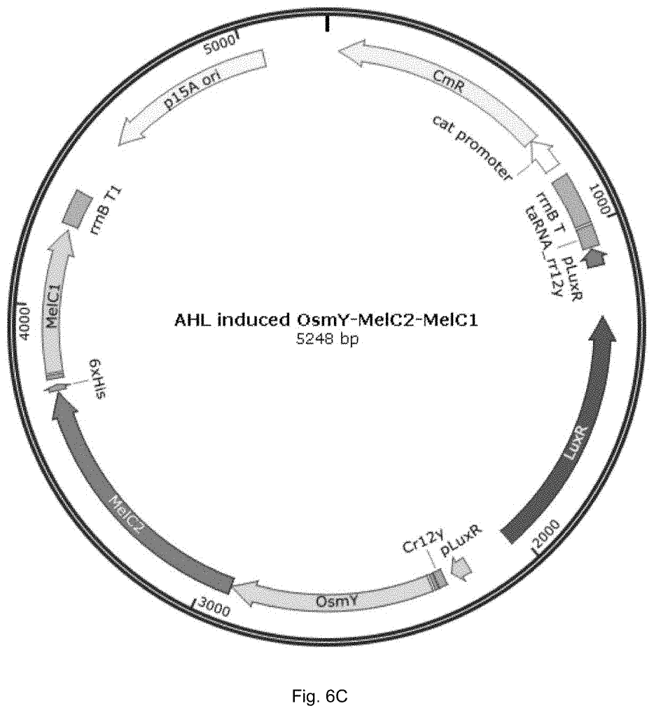

To circumvent these problems, we decided to adopt a “division-of-labor” strategy by introducing an “adhesion-enhancing strain” (AES) into the “glue-producing strain” (GPS) system. The AES was devised for initiating expression and secretion of tyrosinase only in the local environment where the adhesive nanofibrils from cells of the GPS had been massively produced and located. Production of the proteinaceous glue components in the GPS was still regulated by aTc. To enable temporal and spatial control for the production of tyrosinase, we utilized a cellular communication genetic circuit based on the bacterial LuxI-LuxR quorum sensing system.³¹ Specifically, to the GPS, we added an extra aTc-inducible construct coding for the LuxI enzyme, which produced the diffusible cellular communication signal, AHL. The corresponding LuxR protein, together with Lux promoter (pLuxR), was integrated into AES, resulting in AHL-inducible tyrosinase production when culturing these two strains together (FIG. 2A, FIGS. 6A-6C).

Following this division-of-labor strategy, the simultaneous production of protein glue components and enzymes turned out to be in effect under the general culture conditions. TEM, CV, and CR assays all confirmed the successful chemical-triggered production of amyloid nanofibril glue (FIG. 2B). The melanin detection assay together with nitro blue tetrazolium (NBT) staining (samples turn purple because of Dopa or Dopaquinone residues) both indicated remarkable tyrosinase activity in this dual-strain system (FIG. 2C), in agreement with western blot analysis showing the apparent production of the enzyme with appropriate molecular weights in the band. The increasing trends for the catalytic activity and relative expression level of tyrosinase over time (assessed via the time-dependent tyrosinase activity assay and qPCR respectively) further validated the effectiveness of this dual-strain cell-cell communication system during 3 days' incubation. In addition, the similar amounts of biofilm biomass yielded in the dual-strain system compared to that of the GPS alone system implied a negligible level of influence on biofilm growth caused by the AES. Subsequent adhesion measurements (FIG. 2D) revealed that the inclusion of the paired adhesion-enhancing strain in the cultures raised the normalized adhesion force (force/radius, F/R) to 56.6 mN/m. In contrast, the CsgA-Mfp3s strain without its paired strain and the non-induced two-strain system had adhesion force values of 38.1 mN/m and nearly 0 mN/m, respectively (FIG. 2E, 2F). The corresponding lower value for this strain, when cultured without its paired adhesion-enhancing strain, indicated that the secretion of tyrosinase yielded some improvements in adhesive strength.

Having developed the E. coli-based glue-producing system, we next sought to deploy the strains to accomplish specific adhesion tasks upon the detection of environmental stimuli. Initially, we aimed to use the bacteria to capture the floating microspheres from the solution to produce a living composite coating. We cultured the two strains with the green fluorescence-emitting microspheres (d=14 μm) on microscopic glass slides and added inducers to trigger the glue's production (FIG. 2G). After flushing the slides to wash out the non-adhered spheres, the fluorescence microscopy results revealed that aTc-induced glue had trapped a complex and dense collection of microspheres to the glass surfaces, while the controls (non-induced samples) retained few residues (FIG. 2H). Notably, this adhesion persisted even after washing with water for 12 h at a 100 mL/min water flow rate, with most of the objects remaining attached (FIG. 2I). Conceivably, the resultant living composite coatings could be applied as functional coating layers for diverse applications dependent on the functional nature of the engineered microbes and the captured microspheres. For example, engineered microbes bearing both adhesion and other functional features (i.e., the mineralization-promoting or virus-lysis capacities) can be potentially applied for bio-mediated soil improvement,³² living building construction,⁹ and river pathogen treatment.³³ Also, given that the captured microspheres can be further decorated with new functional groups or even replaced with other functional nanomaterials, such as metal-organic frameworks,³⁴ and inorganic functional particles,³⁵ such composite coatings may find broad applications in biocatalysis, artificial photosynthesis, and biomedicine.

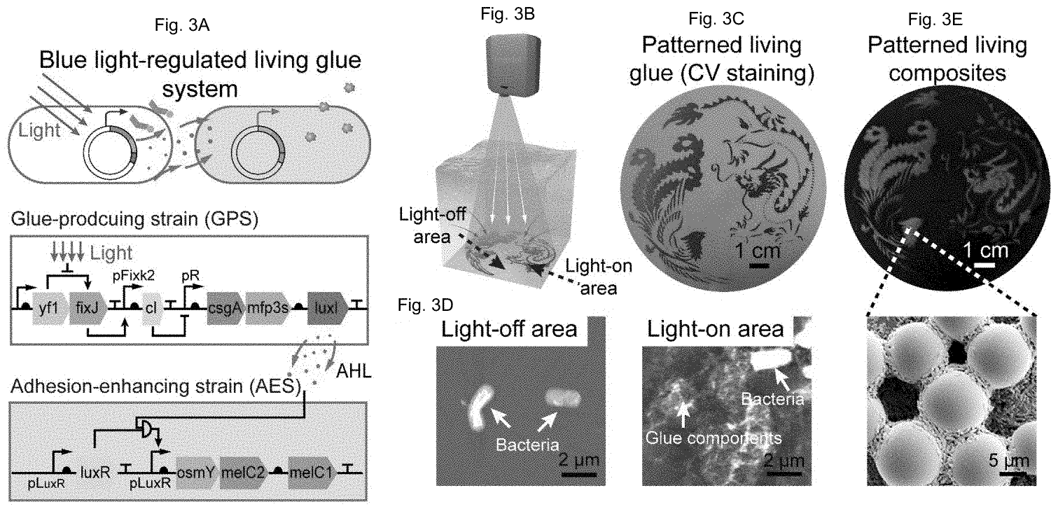

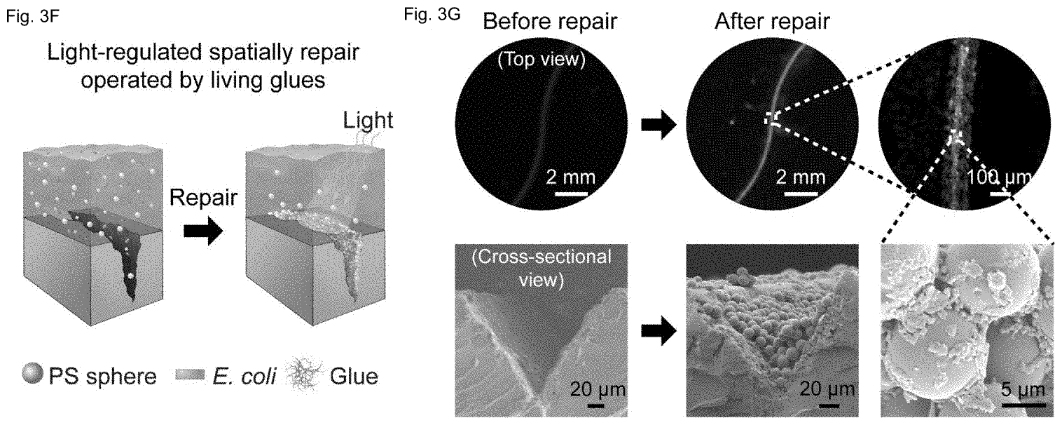

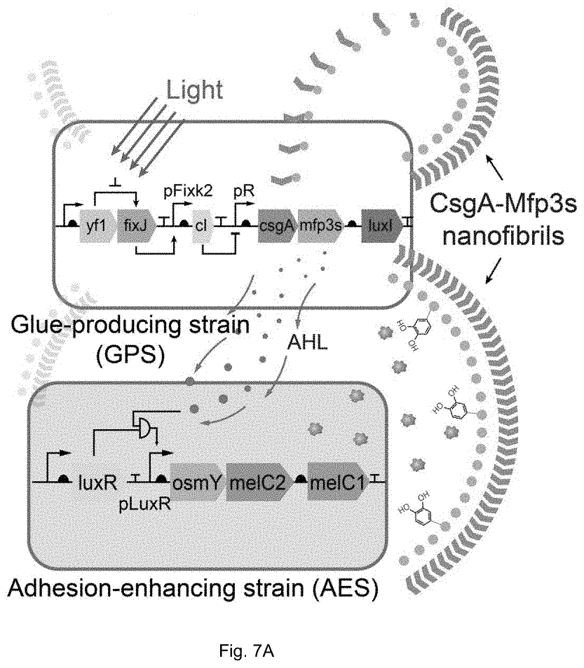

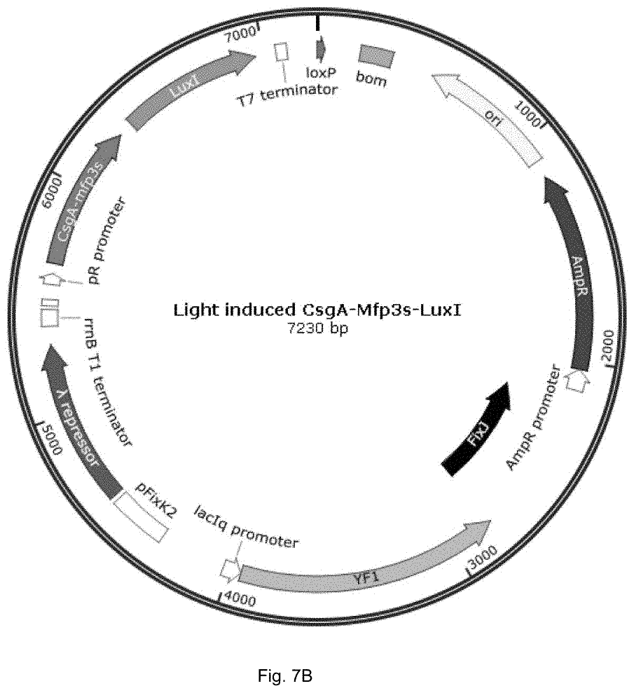

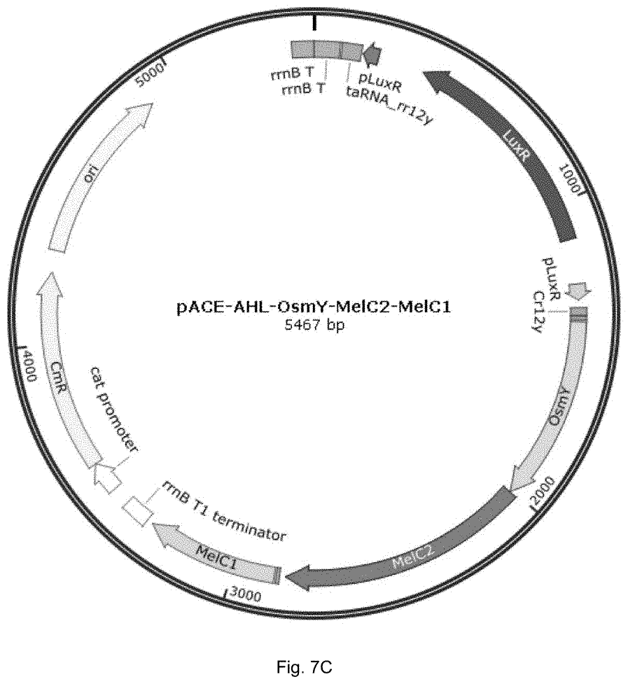

Moving beyond chemical induction and surface coatings, we next developed a spatially-controlled system for precise fabricating living glue and living composite coatings based on responsiveness to light. Noted that the blue light illumination (470 nm) caused negligible influences on microbial growth, we thus replaced the aTc-induced genetic circuit with the well-known pDawn system for blue light-regulated gene expression (FIG. 3A, FIGS. 7A-7C).³⁶ After co-culturing the new strains in the M63 medium, we selectively exposed the cultivated solution to blue light patterns (the Chinese cultural symbols of the dragon and phoenix) emitted from a standard LED projector (FIG. 3B). We found these microbes worked as designed and the glue coatings were only produced at the areas illuminated with blue wavelength (470 nm) light, revealing a clearly defined pattern after CV staining (FIG. 3C) or after thorough binding with red fluorescence-emitting quantum dots through molecular recognition based on “NTA-Metal-His” coordination chemistry.³⁷ AFM image, along with quantitative biomass assessment based on CV and CR assays, revealed that only the illuminated areas of the samples exhibited an extensive amount of nanofibrils, in sharp contrast to the unexposed areas with few protein products (FIG. 3D). We also conducted similar experiments that included living glue-enabled capture of those floating microspheres in the liquid medium and again observed highly spatially resolved patterns comprising a bacteria-sphere hybrid coating (FIG. 3E).

Having demonstrated that our light-regulated glue could predictably implement dynamic underwater adhesions to form spatially resolved patterns on substrates, we next explored the potential of using this living material for active, spatially targeted damage repair work (FIG. 3F). To such ends, we first incubated the bacteria with fluorescent microspheres into an approximately 6 cm long damaged furrow (100 μm wide and 50 μm high) scratched on the culture disk. In theory, upon exposure to light directed to illuminate this furrow, the elicited glue components at the groove should bind the fluorescence-emitting matters together and thereby spatially filled up this gap. Indeed, fluorescent microscopy revealed much stronger fluorescence signals in the furrow areas than non-illuminated areas (FIG. 3G). Regarding damage repair, SEM showed that compared to the original furrow, the light-exposed regions contained much glue components that aggregated the microspheres to each other and the substrate surface—thereby retaining them in place and filling the damage site (FIG. 3G).

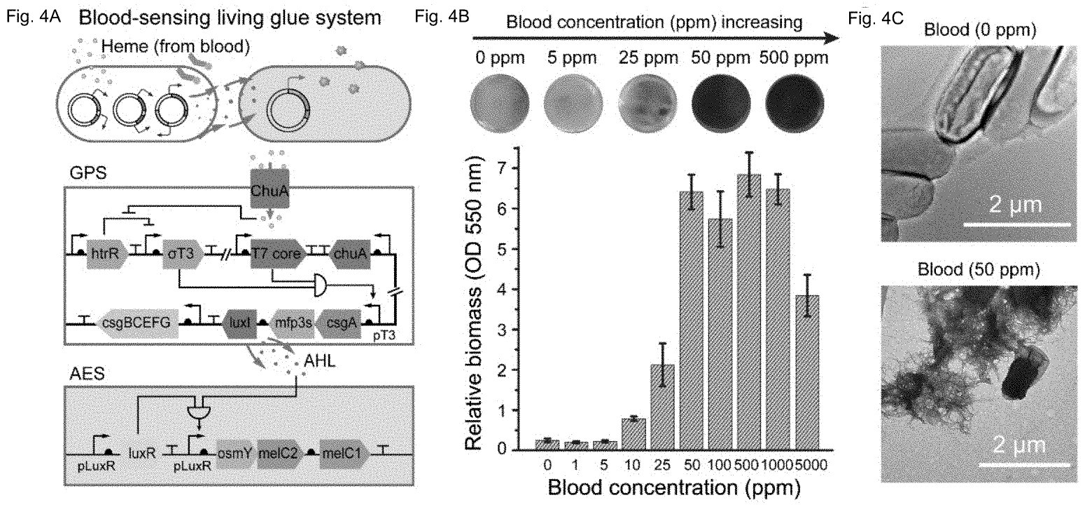

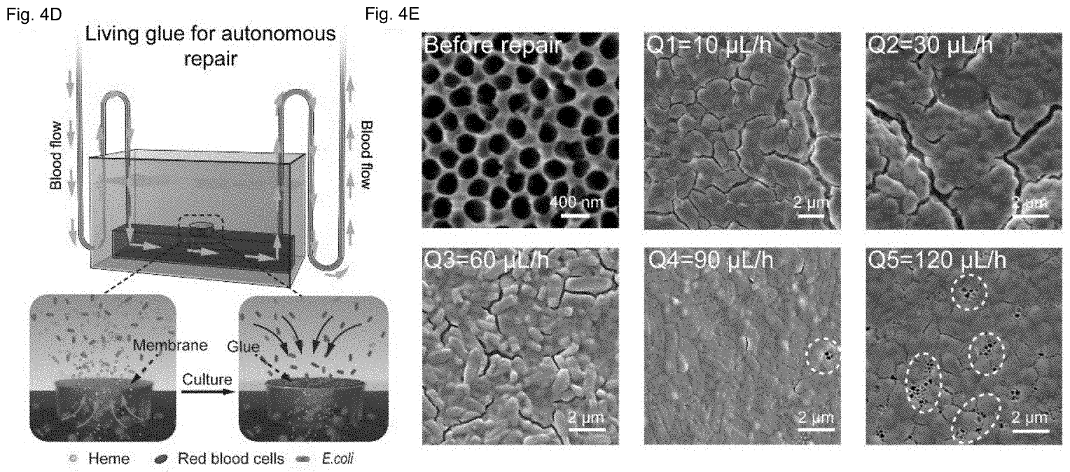

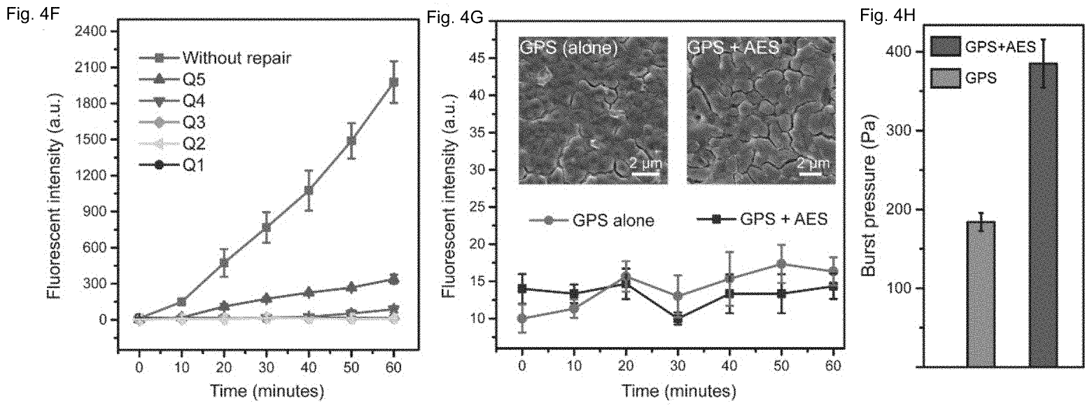

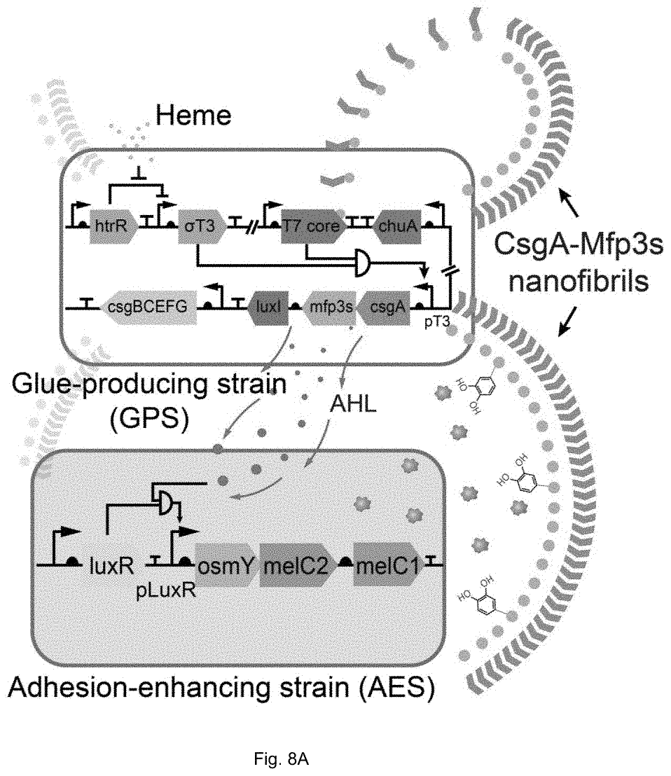

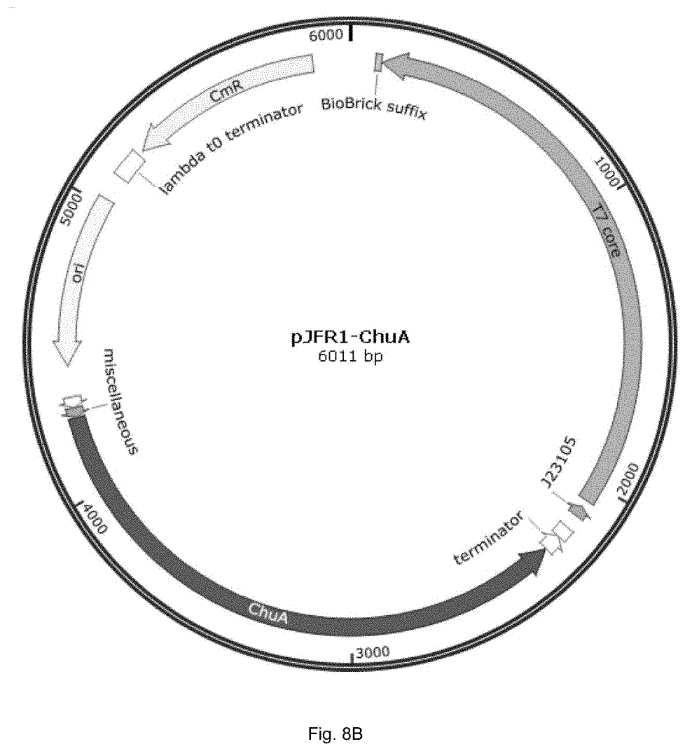

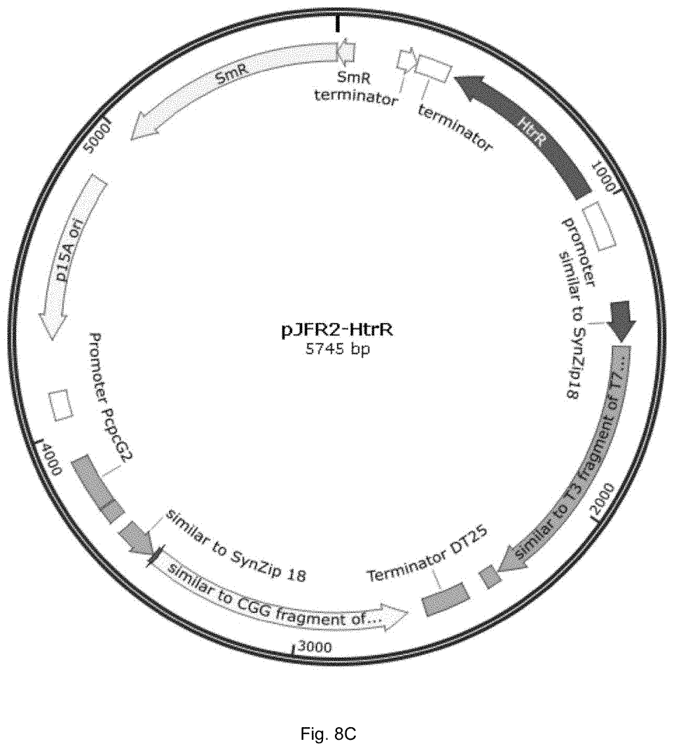

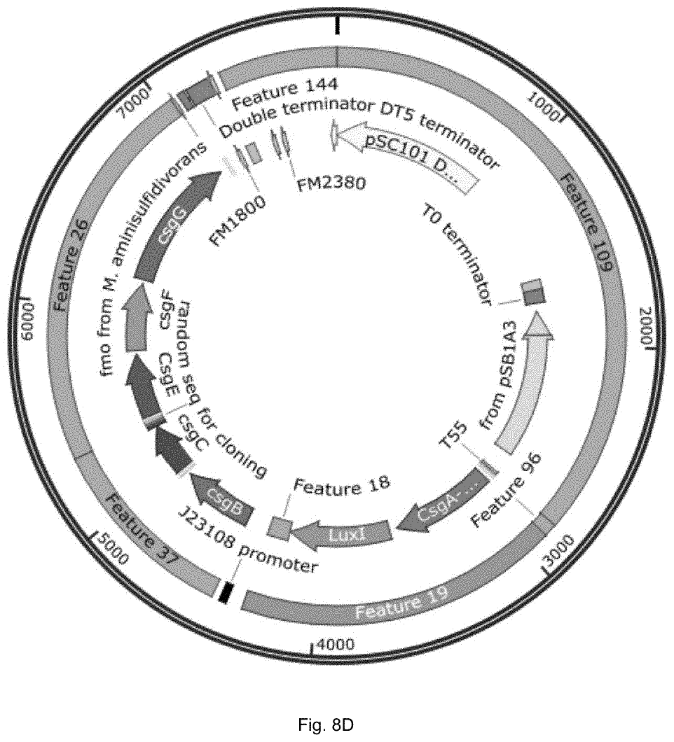

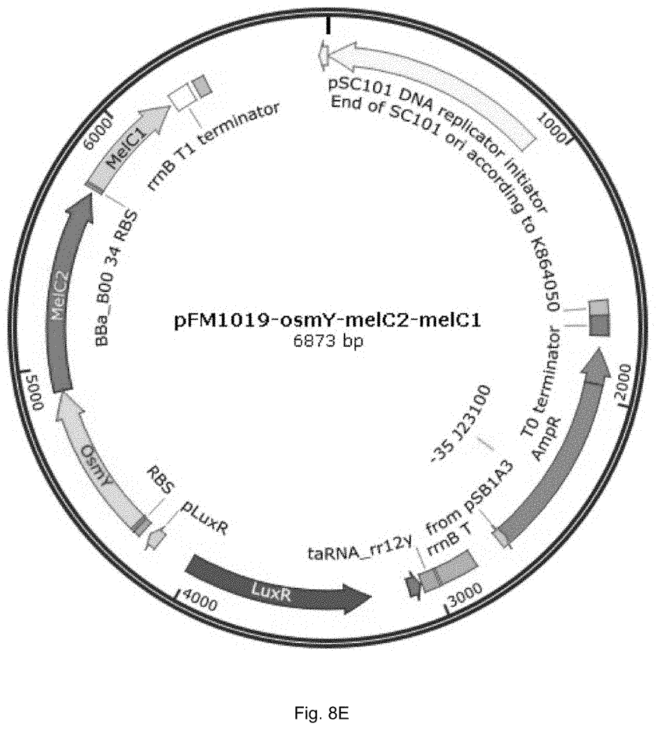

Natural living organisms can self-produce and even possess autonomous repairing features. Motivated by the biological autonomy of living systems, we next turned to build a living glue-enabled autonomous repair system that can sense blood (i.e., heme coordination complexes) and respond by repairing blood leakage sites within a microfluidic device channel. This system aimed to conceptualize a treatment for gastrointestinal bleeding. We maintained the two-strain division-of-labor strategy in our blood-inducible living glue system. However, to demonstrate the self-repairing features, we decided to apply a genetically optimized E. coli as the chassis (E. coli JF1 Δcsg) that allowed fast expression and secretion of corresponding recombinant glue components at above 30° C. (the maximal temperature suited for biofilm secretion).³⁸ We next constructed and transformed a blood-responsive gene circuit based on a blood activation gene network containing the heme transporter (ChuA membrane protein) and heme-sensitive transcriptional repressor HtrR³⁹ into the host strain. This glue-producing cells constitutively expressed the ChuA protein to ensure that heme molecules could be efficiently transported into cells to interact with HtrR, thereby activating T7 polymerase and triggering the expression and secretion of downstream adhesive protein components. Moreover, the GPS also constitutively produced the AHL signal that induced the expression and secretion of tyrosinase by the AES (FIG. 4A, FIGS. 8A-8E). Next, we conducted experiments to test the sensitivity of this blood-induced glue system by varying different concentrations of blood. CV and CR assays both revealed that these living glue materials could be triggered by blood at levels as low as ten parts per million (ppm). They reached maximum production at above 50 ppm blood (FIG. 4B), as observed by TEM showed large amounts of nanofibrils surrounding individual cells (FIG. 4C). Notably, the bacteria samples grew in the absence of blood exhibited almost no nanofibrils formation (FIG. 4C), implying the rigorousness of our operating system.

Having established the ability for blood-induced glue production, we next turned to develop a living system capable of autonomous damage repair. Pursuing this idea, we devised an experimental setup to mimic a slightly damaged bleeding vascular tissue based on a microfluidic device, and red blood cells (FIG. 4D). The channel of the microfluidic device was 2.0 cm long, 200 μm wide, and 80 μm high in dimension and was connected to an input and output tubing through which horse blood could be pumped. For damage sites, we created a small hole (d=1.6 mm) in the microfluidic channel, which was then covered by an alumina membrane containing a network of pores (d=400 nm for each pore). Given the pore size, we anticipated that this damaged device would enable the leakage of heme into the culture medium, where the blood-sensing and paired adhesion-enhancing strains were cultured. Ideally, the engineered strains would actively localize to the damage site, and plug up all the 400 nm pores of the simulated rupture site (FIG. 4D).

When the microfluidic devices pumped with horse blood (at a blood flow, Q=10 μL/hour) were immersed in the dual-strain culture solution, living glue productions were found to be successfully triggered on the leaking membrane surfaces. The living glues filled in the pores and formed robust coatings covering the damaged sites, as revealed by SEM (FIG. 4E). In contrast, the membrane surfaces of the leaking site had only been contaminated with a sparse amount of bacterial in the control groups (microfluidic devices without pumped blood repaired under the same culture solution). The SEM results thus clearly indicated the noticeable repairing effect of living glues upon exposure to leaking blood. To further assess whether the blood flow rate would affect the coating morphology and repairing effects, we performed similar repairing experiments under different flows ranging from 10 to 120 μL/hour. We found that, despite coating formation occurring in all cases, an increasing amount of coating defects started to appear on the coating surfaces as the blood flow exceeded 60 μL/hour, which might be ascribed to the higher flow pressure locally impeding bacterial aggregation and biofilm formation (FIG. 4E).

Next, we conducted leakage experiments before and after the glue-mediated autonomous repair to evaluate the robustness of the seal. For quantitative detection, we used the Cy3 molecule as a fluorescent marker: Cy3 has a specific linear concentration-fluorescence relationship and is of similar molecular weight with heme (MW=616.5). A 50 μM Cy3 solution was pumped through the broken and subsequently repaired the microfluidic device with a fast flow rate (250 μL/hour). The fluorescence intensity of Cy3 in PBS buffer in the surrounding solution was measured periodically (at every 10-min interval). The leakage experiments indicated that fluorescence leakages were found associated with those living glue-repaired samples performed under blood flow exceeding 60 μL/hour (FIG. 4F), in consistence with the aforementioned SEM observations showing that coating defects started to form on the porous damage sites once blood flow exceeded 60 μL/hour (FIG. 4E). These results thus demonstrated that our blood-sensing living glue could successfully perform autonomous repair work at low and intermediate blood flow speeds.

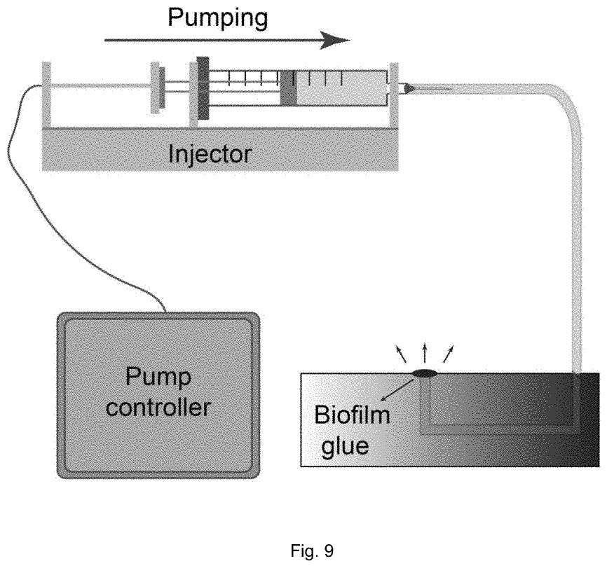

To validate if the AES would indeed enhance the repairing effect in our dual-strain living glue system, we performed additional tests for bleeding-induced repairs in the presence of GPS alone (at a blood flow of 10 μL/hour). SEM morphological characterization and fluorescence leakage tests indicated that similar to the dual-strain glue system (GPS+AES), GPS alone was able to form dense biofilm materials on the porous surfaces of the damage sites and could block the leakage of the fluorescent dye solution (FIG. 4G). However, both the adhesion performance and burst pressure resistance of the dual-strain system were superior to those of the GPS alone system. Specifically, the dual-strain glue system exhibited a substantially higher level of adhesion energy (4.38±0.36 mJ/m²) than that of the GPS alone system (2.64±0.36 mJ/m²), as measured by AFM colloidal probe technique. For the burst pressure resistance tests (FIG. 9), the dual-strain glue system could resist about 385±30 Pa pressure, in stark contrast with the much lower pressure resistance for the GPS alone system (184±11 Pa) (FIG. 4H). Taken together, these results again validated the effectiveness of the division-of-labor strategy for our living glue design and also indicated that the presence of AES in the bleeding-induced glue system enhanced the performances of GPS in terms of adhesion strength and mechanical robustness. Despite those advantages of the dual-strain system, the more biocompatible GPS alone system, operating without the use of tyrosinase and thus high Cu²⁺ concentration, might be a better option for in vivo applications that do not require higher mechanical properties.

Previous studies suggest that the solidification of barnacle amyloid adhesive could be regarded as a specific wound healing process, in which nanofibrils assembly and crosslinking into networks share similarities to the blood clotting.⁴⁰ Similarly, our bio-inspired living glues, to some extent, mimicked the natural blood-clotting process:⁴¹ the blood-sensing bacterial firstly sense and migrate on the damaged sites to form a weak plug (similar to the formation of platelet plug) and then secrete adhesive amyloid nanofiber glues to stabilize the plug (similar to the formation of blood clots). Our systems can borrowed from biology to engineer even smarter and more efficient living glue systems. For example, the amyloid component in our system can be functionalized with fibrinogen-derived RGD-containing protein domains or peptides to generate more biocompatible glues. Additionally, glue performance can be augmented by genetically controlling the thrombin's secretion supplemented with the adhesive amyloid nanofibrils.

Discussion

Ever-deepening understanding of the robust biological adhesion systems of marine organisms has dramatically advanced the development of bio-inspired adhesives, including various small molecule-based, polymeric, and proteinaceous adhesives.⁴²⁻⁴⁶ Despite their impressive adhesion performances, such bio-inspired adhesives lack the “living” attributes of natural adhesive systems that inspired them. Taking one step further, we previously demonstrated a Bacillus subtilis biofilm glue with regenerative capacity and environmental tolerance.²² However, this living glue did not exploit the full potential of living systems to respond autonomously to environmental stimuli, as marine adhesive systems do. Here, we have developed environmentally responsive living glue systems as a new type of adhesive that can perform diverse on-demand mechanical operation tasks, including capturing non-sticky microspheres from solution to form living composite coatings and performing targeted repair in a spatially controlled manner. Furthermore, we showed that these systems could be programmed to perform adhesion repairs by autonomously sensing stimuli (blood) and repairing leaking pores in a microfluidic device.

### REFERENCES

1. Nguyen, P. Q., Courchesne, N. D., Duraj-Thatte, A., Praveschotinunt, P. & Joshi, N. S. (2018). Engineered Living Materials: Prospects and Challenges for Using Biological Systems to Direct the Assembly of Smart Materials. Adv Mater 30, e1704847.
2. Gilbert, C. & Ellis, T. (2019). Biological Engineered Living Materials: Growing Functional Materials with Genetically Programmable Properties. Acs Synth Biol 8, 1-15.
3. Chen, A. Y., Zhong, C. & Lu, T. K. (2015). Engineering Living Functional Materials. Acs Synth Biol 4, 8-11.
4. Ball, P. (2018). Synthetic biology—Engineering nature to make materials. MRS Bulletin 43, 477-484.
5. Chen, A. Y. et al. (2014). Synthesis and patterning of tunable multiscale materials with engineered cells. Nat Mater 13, 515-523.
6. Nguyen, P. Q., Botyanszki, Z., Tay, P. K. R. & Joshi, N. S. (2014). Programmable biofilm-based materials from engineered curli nanofibres. Nat Commun 5, 4945.
7. Cao, Y. et al. (2017). Programmable assembly of pressure sensors using pattern-forming bacteria. Nat Biotechnol 35, 1087-1093.
8. Huang, J. et al. (2019) Programmable and printable Bacillus subtilis biofilms as engineered living materials. Nat Chem Biol 15, 34-41.
9. Heveran, C. M. et al. (2020). Biomineralization and Successive Regeneration of Engineered Living Building Materials. Matter 2, 481-494.
10. Florea, M. et al. (2016). Engineering control of bacterial cellulose production using a genetic toolkit and a new cellulose-producing strain. Proceedings of the National Academy of Sciences 113, E3431-E3440.
11. Gilbert, C. et al. (2019). Living materials with programmable functionalities grown from engineered microbial co-cultures. bioRxiv, 2019.2012.2020.882472, doi:10.1101/2019.12.20.882472.
12. Liu, X. et al. (2018). 3D Printing of Living Responsive Materials and Devices. Adv Mater 30, 1704821.
13. Tang, T.-C. et al. (2020). Tough Hydrogel-Based Biocontainment of Engineered Organisms for Continuous, Self-Powered Sensing and Computation. bioRxiv, 2020.2002.2011.941120, doi:10.1101/2020.02.11.941120.
14. Sun, G. L., Reynolds, E. E. & Belcher, A. M. (2020). Using yeast to sustainably remediate and extract heavy metals from waste waters. Nature Sustainability 3, 303-311.
15. Bourdeau, R. W. et al. (2018). Acoustic reporter genes for noninvasive imaging of microorganisms in mammalian hosts. Nature 553, 86-90.
16. González, L. M., Mukhitov, N. & Voigt, C. A. (2020). Resilient living materials built by printing bacterial spores. Nat Chem Biol 16, 126-133.
17. Dai, Z. et al. (2019). Versatile biomanufacturing through stimulus-responsive cell-material feedback. Nat Chem Biol 15, 1017-1024.
18. Schaffner, M., Rühs, P. A., Coulter, F., Kilcher, S. & Studart, A. R. (2017). 3D printing of bacteria into functional complex materials. Science Advances 3, eaao6804.
19. Hay, J. J. et al. (2018). Bacteria-Based Materials for Stem Cell Engineering. Adv Mater 30, 1804310.
20. Callura, J. M., Cantor, C. R. & Collins, J. J. (2012). Genetic switchboard for synthetic biology applications. Proceedings of the National Academy of Sciences 109, 5850-5855.
21. Meyer, A. J., Segall-Shapiro, T. H., Glassey, E., Zhang, J. & Voigt, C. A. (2019). Escherichia coli “Marionette” strains with 12 highly optimized small-molecule sensors. Nat Chem Biol 15, 196-204.
22. Zhang, C. et al. (2019). Engineered Bacillus subtilis biofilms as living glues. Materials Today 28, 40-48.
23. Stewart, R. J., Ransom, T. C. & Hlady, V. (2011). Natural Underwater Adhesives. J Polym Sci Pol Phys 49, 757-771.
24. Waite, J. H. (2017). Mussel adhesion-essential footwork. J Exp Biol 220, 517-530.
25. Stewart, R. J., Wang, C. S., Song, I. T. & Jones, J. P. (2017). The role of coacervation and phase transitions in the sandcastle worm adhesive system. Adv Colloid Interface Sci 239, 88-96.
26. Chapman, M. R. et al. (2002). Role of Escherichia coli curli operons in directing amyloid fiber formation. Science 295, 851-855.
27. Cui, M. et al. (2019). Exploiting mammalian low-complexity domains for liquid-liquid phase separation-driven underwater adhesive coatings. Science Advances 5, eaax3155.
28. Zhong, C. et al. (2014). Strong underwater adhesives made by self-assembling multi-protein nanofibres. Nat Nanotechnol 9, 858-866.
29. Kohashi, P. Y. et al. (2004). An efficient method for the overexpression and purification of active tyrosinase from Streptomyces castaneoglobisporus. Protein Expr Purif 34, 202-207.
30. Kotzsch, A. et al. (2011). A secretory system for bacterial production of high-profile protein targets. Protein Sci 20, 597-609.
31. Fuqua, W. C., Winans, S. C. & Greenberg, E. P. (1994). Quorum sensing in bacteria: the LuxR-LuxI family of cell density-responsive transcriptional regulators. Journal of bacteriology 176, 269-275.
32. DeJong, J. T., Mortensen, B. M., Martinez, B. C. & Nelson, D. C. (2010). Bio-mediated soil improvement. Ecological Engineering 36, 197-210.
33. Pu, J. et al. (2020). Virus Disinfection from Environmental Water Sources Using Living Engineered Biofilm Materials. Advanced Science 7, 1903558.
34. Ji, Z., Zhang, H., Liu, H., Yaghi, O. M. & Yang, P. (2018). Cytoprotective metal-organic frameworks for anaerobic bacteria. Proceedings of the National Academy of Sciences 115, 10582-10587.
35. Lee, H. P. & Gaharwar, A. K. Light-Responsive Inorganic Biomaterials for Biomedical Applications. Advanced Science n/a, 2000863, doi:10.1002/advs.202000863.
36. Ohlendorf, R., Vidayski, R. R., Eldar, A., Moffat, K. & Möglich, A. (2012). From Dusk till Dawn: One-Plasmid Systems for Light-Regulated Gene Expression. J Mol Biol 416, 534-542.
37. Wang, X. et al. (2018). Programming Cells for Dynamic Assembly of Inorganic Nano-Objects with Spatiotemporal Control. Adv Mater 30, e1705968.
38. Moser, F., Tham, E., Gonzalez, L. M., Lu, T. K. & Voigt, C. A. (2019). Light-Controlled, High-Resolution Patterning of Living Engineered Bacteria Onto Textiles, Ceramics, and Plastic. Adv Funct Mater 29, 1901788.
39. Mimee, M. et al. (2018). An ingestible bacterial-electronic system to monitor gastrointestinal health. Science 360, 915-918.
40. Dickinson, G. H. et al. (2009). Barnacle cement: a polymerization model based on evolutionary concepts. J Exp Biol 212, 3499-3510.
41. Periayah, M. H., Halim, A. S. & Mat Saad, A. Z. (2017). Mechanism Action of Platelets and Crucial Blood Coagulation Pathways in Hemostasis. Int J Hematol Oncol Stem Cell Res 11, 319-327.
42. Rose, S. et al. (2014). Nanoparticle solutions as adhesives for gels and biological tissues. Nature 505, 382-385.
43. Maier, G. P., Rapp, M. V., Waite, J. H., Israelachvili, J. N. & Butler, A. (2015). Adaptive synergy between catechol and lysine promotes wet adhesion by surface salt displacement. Science 349, 628-632.
44. Li, J. et al. (2017). Tough adhesives for diverse wet surfaces. Science 357, 378-381.
45. Baik, S. et al. (2017). A wet-tolerant adhesive patch inspired by protuberances in suction cups of octopi. Nature 546, 396-400.
46. Annabi, N. et al. (2017). Engineering a highly elastic human protein-based sealant for surgical applications. Science Translational Medicine 9, eaai7466.

1. A living engineered glue system for performing autonomous mechanical repairs, the system comprising a biofilm of microbial cells embedded in an extracellular matrix and operably linked in an environmentally-inducible, cell-cell communication genetic circuit to control gene expression, the cells comprising:
a glue-producing strain secreting a signal molecule and expressing a fusion protein comprising an adhesive domain and a biofilm protein domain, wherein expression of the fusion protein is induced by an environmental inducer; and
an adhesion enhancing strain expressing a tyrosinase, wherein expression of the tyrosinase is induced by the signal molecule secreted by the glue-producing strain.

2. The system of claim 1, wherein the adhesive domain is selected from a marine organism protein adhesive domain (such as a mussel foot protein domain or a barnacle amyloid adhesive domain), a metal-binding peptides/protein domain, minerals-binding peptide/protein domain, and a trefoil factor family (TFF) protein domain.

3. The system of claim 1, wherein the adhesive domain comprises a mussel foot protein domain selected from a Mfp3, Mfp3s, Mfp5, Mfp8, and Mfp3s-derived peptide.

4. The system of claim 1, wherein the biofilm is selected from an E. coli biofilm (CsgA-based), a B. subtilis biofilm (TasA-based), a kombucha biofilm (acetic acid bacteria (Acetobacteraceae) and osmophilic yeast), and a yeast biofilm (Sup35 amyloid protein-based).

5. The system of claim 1, wherein the adhesive domain comprises a mussel foot protein domain selected from a Mfp3, Mfp3s, Mfp5, Mfp8, and Mfp3s-derived peptide, and
the biofilm is selected from an E. coli biofilm (CsgA-based), a B. subtilis biofilm (TasA-based), a kombucha biofilm (acetic acid bacteria (Acetobacteraceae) and osmophilic yeast), and a yeast biofilm (Sup35 amyloid protein-based).

6. The system of claim 1, wherein the biofilm protein domain is selected from: TasA (B. subtilis), CsgA (E. coli), PSMs (S. aureus), RmbC (V. cholera), CsgA (Enterobacter cloacae), FapC (Pseudomonas spp.), CsgA (Salmonella spp.) or PAc (Streptococcus mutans).

7. The system of claim 1, wherein the adhesive domain comprises a mussel foot protein domain selected from a Mfp3, Mfp3s, Mfp5, Mfp8, and Mfp3s-derived peptide, and
the biofilm protein domain is selected from: TasA (B. subtilis), CsgA (E. coli), PSMs (S. aureus), RmbC (V. cholera), CsgA (Enterobacter cloacae), FapC (Pseudomonas spp.), CsgA (Salmonella spp.) or PAc (Streptococcus mutans).

8. The system of claim 1, wherein the biofilm protein domain comprises a CsgA monomer.

9. The system of claim 1, wherein the adhesive domain comprises a mussel foot protein domain selected from a Mfp3, Mfp3s, Mfp5, Mfp8, and Mfp3s-derived peptide, and
the biofilm protein domain comprises a CsgA monomer.

10. The system of claim 1 wherein the environmental inducer is selected from a blood component (e.g. heme), light (e.g. blue/red/green light), heat/thermal, salt/electrolyte concentration, pH, electrons, and small signal molecules such as isopropyl-beta-D-thiogalactoside (IPTG), anhydrotetracycline (aTC), bile acid or thiosulfate.

11. The system of claim 1, wherein the genetic circuit provides a sensor for, and is environmentally-responsive to a signal selected from: aTc/blue light, green/red light, blood/heme, thermal/heat, pH, salt concentration, IPTG, bile acid, thiosulfate, and electrons.

12. The system of claim 1 wherein the environmental inducer is selected from a blood component (e.g. heme), light (e.g. blue/red/green light), heat/thermal, salt/electrolyte concentration, pH, electrons, and small signal molecules such as isopropyl-beta-D-thiogalactoside (IPTG), anhydrotetracycline (aTC), bile acid or thiosulfate, and
the genetic circuit provides a sensor for, and is environmentally-responsive to a signal selected from: aTc/blue light, green/red light, blood/heme, thermal/heat, pH, salt concentration, IPTG, bile acid, thiosulfate, and electrons.

13. The system of claim 5 wherein the environmental inducer is selected from a blood component (e.g. heme), light (e.g. blue/red/green light), heat/thermal, salt/electrolyte concentration, pH, electrons, and small signal molecules such as isopropyl-beta-D-thiogalactoside (IPTG), anhydrotetracycline (aTC), bile acid or thiosulfate, and
the genetic circuit provides a sensor for, and is environmentally-responsive to a signal selected from: aTc/blue light, green/red light, blood/heme, thermal/heat, pH, salt concentration, IPTG, bile acid, thiosulfate, and electrons.

14. The system of claim 7 wherein the environmental inducer is selected from a blood component (e.g. heme), light (e.g. blue/red/green light), heat/thermal, salt/electrolyte concentration, pH, electrons, and small signal molecules such as isopropyl-beta-D-thiogalactoside (IPTG), anhydrotetracycline (aTC), bile acid or thiosulfate, and
the genetic circuit provides a sensor for, and is environmentally-responsive to a signal selected from: aTc/blue light, green/red light, blood/heme, thermal/heat, pH, salt concentration, IPTG, bile acid, thiosulfate, and electrons.

15. The system of claim 1, wherein the microbial cells are selected from: Bacillus spp. (e.g. B. subtilis), Pseudomonas spp. (e.g. P. aeruginosa), Staphylococcus spp. (e.g. S. aureus), Salmonella ssp. (e.g. S. enterica), Vibrio spp. (e.g. V. cholera), Streptococcus spp. (e.g. Streptococcus mutans), Enterobacter spp. (e.g. Enterobacter cloacae), Lactobacillus spp. (e.g. L. plantarum) or Escherichia spp. (e.g. E. coli).

16. The system of claim 5, wherein the microbial cells are selected from: Bacillus spp. (e.g. B. subtilis), Pseudomonas spp. (e.g. P. aeruginosa), Staphylococcus spp. (e.g. S. aureus), Salmonella ssp. (e.g. S. enterica), Vibrio spp. (e.g. V. cholera), Streptococcus spp. (e.g. Streptococcus mutans), Enterobacter spp. (e.g. Enterobacter cloacae), Lactobacillus spp. (e.g. L. plantarum) or Escherichia spp. (e.g. E. coli).

17. The system of claim 7, wherein the microbial cells are selected from: Bacillus spp. (e.g. B. subtilis), Pseudomonas spp. (e.g. P. aeruginosa), Staphylococcus spp. (e.g. S. aureus), Salmonella ssp. (e.g. S. enterica), Vibrio spp. (e.g. V. cholera), Streptococcus spp. (e.g. Streptococcus mutans), Enterobacter spp. (e.g. Enterobacter cloacae), Lactobacillus spp. (e.g. L. plantarum) or Escherichia spp. (e.g. E. coli).

18. The system of claim 9, wherein the microbial cells are selected from: Bacillus spp. (e.g. B. subtilis), Pseudomonas spp. (e.g. P. aeruginosa), Staphylococcus spp. (e.g. S. aureus), Salmonella ssp. (e.g. S. enterica), Vibrio spp. (e.g. V. cholera), Streptococcus spp. (e.g. Streptococcus mutans), Enterobacter spp. (e.g. Enterobacter cloacae), Lactobacillus spp. (e.g. L. plantarum) or Escherichia spp. (e.g. E. coli).

19. A method of using the living glue system of claim 1, for performing autonomous mechanical repairs to a surface of a mechanical device or component thereof, such as sealing a defect, comprising the step of: providing the surface coated with the system or applying the system to the surface, under conditions wherein the system autonomously senses and repairs the defect.

20. A method of making the living glue system of claim 1, comprising engineering and/or combining the glue-producing strain and the adhesion enhancing strain to form the system.
